(19) United States
(12) Patent Application Publication
(10) Pub. No.: US 2008/0097241 A1
(43) Pub. Date: Apr. 24, 2008

(54) SAMPLING DEVICE

(75) Inventors: George MALTEZOS, Fort Salonga, NY (US); Axel Scherer, Lagona Beach, CA (US)

Correspondence Address: WILSON SONSINI GOODRICH & ROSATI, 650 PAGE MILL ROAD, PALO ALTO, CA, 94304-1050, US

(73) Assignee: California Institute of Technology

(21) Appl. No.: 11/874,830

(22) Filed: Oct. 18, 2007

Related U.S. Application Data
(60) Provisional application No. 60/852,830, filed on Oct. 18, 2006.

(51) Int. Cl. A61B 5/151

(52) U.S. Cl. 600/576; 600/583

(57) ABSTRACT

This invention provides devices and methods for obtaining a sample.

### CROSS-REFERENCE

This application claims the benefit of U.S. Provisional Application No. 60/852,830 filed Oct. 18, 2006, which is incorporated herein by reference.

### BACKGROUND OF THE INVENTION

Sampling devices have been developed to obtain body fluids, such as blood for testing and analysis. Generally, lancets are used to pierce the skin, to allow for the collection of a blood sample from the opening created. The blood is transferred to a test device or collection device. Blood is most commonly taken from the fingertips, where the supply is quite good. However, alternate sites, such as the heel and limbs, may also provide areas for drawing bodily fluids.

To reduce the anxiety of piercing the skin and the associated pain, many spring loaded devices have been developed.

For example, U.S. Pat. Nos. 4,503,856, 4,517,978, 4,920,977 describes a spring loaded lancet sampling devices, including single use devices (U.S. Pat. Nos. 4,360,016, 4,924,879). Furthermore, U.S. Pat. Nos. 5,368,047, 4,653,513, 5,320,607 each describe suction-type blood samplers.

However, there remains a need to sampling devices which efficiently obtain samples, such as bodily fluids.

### SUMMARY OF THE INVENTION

Methods and devices are provided for obtaining a sample from a subject. Devices and methods of the invention for obtaining a sample comprise a capillary tube which can be used to obtain a sample from a subject by the capillary tube contacting and breaking a skin surface, contacting and collecting a sample from a body part/cavity (e.g., buccal, vaginal, nasal), or a capillary tube comprising a coaxially situated cutting/piercing implement.

A further aspect of the devices and methods of invention is directed to a capillary tube and coaxial cutting/piercing implement provided as an assembly which can be coupled to an impulse component. Impulse components comprise at least one or more impulse means to provide a jabbing forward of the cutting/piercing implement, so as to extend and retract said implement to break a skin surface to draw a sample, including but not limited to a blood sample.

In a yet further aspect of the invention devices of the invention can be coupled to additional components/devices subsequent to sample collection so as to provide downstream processing or analysis of the sample collected.

In various embodiments of the invention, a capillary tube is coated with one or more reagents as further described herein.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a capillary and pin assembly; 100: pin/needle/wire/lance; 101: impulse means housing unit; 102: capillary tube; 103: shroud/sleeve; 104: finger; 105: finger nail.

FIG. 2 illustrate a dermal layer of skin.

### DETAILED DESCRIPTION OF THE INVENTION

Generally this invention is directed to methods and devices for providing automated or semi-automated sampling from a subject. In various embodiments, a device is provided comprising a capillary tube comprising a hollow lumen in which is present an implement for cutting or piercing skin to draw a sample from a subject. The implement for cutting or piercing can be a pin, wire, needle or lancet as further described herein. Furthermore, an optional sleeve or shroud can provide a protective barrier to prevent or reduce the likelihood of contamination.

Device

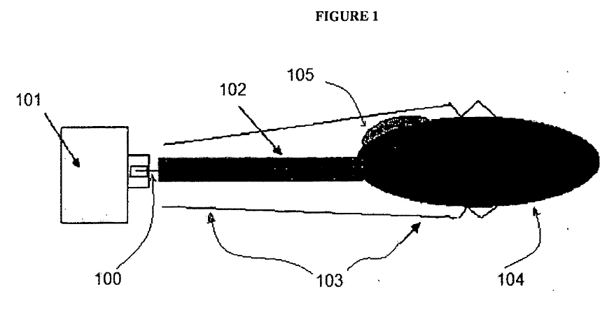

The device FIG. 1 is meant for use as an automated or semi-automated device to draw blood from a patients finger 104 or heel or other area. The device can protect the patient and the sample from cross contamination, and also protect a person drawing a sample (e.g., blood), from coming in contact with the blood. However, in some embodiments, no such person is necessary since the operation of the device is automatic (e.g., controlled by computer executable logic which functions to sense contact with a subject's skin, which triggers activation of the device) or is triggered by the subject activating the device through a switch controlling activation of the device.

In one embodiment, a device of the invention is also meant as a first stage to collecting blood for a microfluidic direct assay or sample preparation device.

Capillary Assembly

In some embodiments, a capillary tube 102 of the invention can be of the type that contains an anti-coagulant on the walls of the tube to prevent the blood from clotting.

In various embodiments of the invention, a capillary tube 102 can be made of material including but not limited to glass, glass-like material, polypropylene, polycarbonate, acrylonitrile, butadiene, styrene, polyvinyl chloride or some other similar material that is able to maintain the sub-atmospheric pressure created during use. In one embodiment, a capillary tube is comprised of glass or a glass like material.

In various embodiments, a capillary tube of the invention has a diameter of about size range is about 0.25 mm to about 5 mm, about 0.5 mm to about 2.5 mm, about 1 mm to about 5 mm, about 2.0 mm to about 5 mm, or about 3 mm to about 5 mm; and a length of about 0.6 mm to about 130 mm, about 0.6 to 20 mm, about 5 mm to about 50 mm, about 25 mm to about 75 mm, about 40 mm to about 100 mm, about 60 mm to about 130 mm, about 80 mm to about 130 mm, about 100 mm to about 130 mm, or about 0.5 mm to about 10 mm.

Therefore, in some embodiments, a capillary tube can be selected, manufactured within any of these ranges to provide a particular capacity. For example, the diameter can be about 0.25, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or 5.0 mm. Furthermore, for any given diameter, the length of a capillary tube can be selected to be about 6, about 6.1, about 6.2, about 6.3, about 6.4, about 6.5, about 6.6, about 6.7, about 6.8, about 6.9, about 7.0, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 121, about 122, about 123, about 124, about 125, about 126, about 127, about 128, about 129 or 130 mm. The term “about” means that measures of less/greater than 10% are included herein. Furthermore, such sizing can be utilized with any capillary tube described herein.

Furthermore, in various embodiments, the cutting/piercing implement 100 can be longer or shorter than the capillary tube. For example, where the implement is longer, a certain length will stick out past the distal end of the capillary tube and will fit into a adaptor or seat positioned on the impulse means housing unit 101 such that the capillary tube is held in place (such as through a luer lock or friction fit, or other means known in the art). The impulse means (e.g., spring actuator). Lancing/cutting assemblies are well-known in the art. Representative examples of lancing assemblies suitable for this invention are described in U.S. Pat. Nos. Re. 32,922, 4,203,446, 4,990,154, and 5,487,748. Any lancing/cutting assembly selected should operate in conjunction with the other features of the apparatus of this invention. For example, if a vacuum is employed, the lancing assembly must be designed so that a vacuum can be formed and drawn through the assembly. The lancing assembly can be designed to allow automatic cocking and automatic triggering of the lancet. Therefore, in one embodiment a coaxial impulse means impels (jabs) the piercing/cutting implement forward, and the implement subsequently retracts. The term “jab” or “jabbing” means the implement is thrust forwarded to a defined length/depth (e.g., as set by a mechanical stop) and retracts. Typically such jabbing motion is complete in one second or less (0.1, 0.2, 0.3, 0.4, 0.5, 0.6, 0.7, 0.8, 0.9 seconds).

While conventional lancing assemblies are suitable for use in this invention, a lancing assembly that utilizes differential gas pressure to thrust a lancet into skin tissue has been developed for use with this invention. As used herein, the expression “differential gas pressure” means the difference in gas pressure between a gas source at a high pressure, e.g., ambient air or pressurized air, and a gas source at a low pressure, e.g., air within a vacuum. In any event, the pressure of a gas source at high pressure exceeds the pressure of a gas source at low pressure.

In various embodiments, a capillary tube 102 of the invention (including any displacement by the cutting or piercing implement 100) has a capacity of from about 0.010 μl to about 1000 μl. In some embodiments, such capacity is about 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl, 43 μl, 44 μl, 45 μl, 46 μl, 47 μl, 48 μl, 49 μl or 50 μl.

In some embodiments, the capillary tube 102 capacity is from about 50 μl to about 250 μl, about 100 μl to about 300 μl, about 250 μl to about 500 μl, about 400 μl to about 750 μl, about 500 μl to about 800 μl, or 750 μl to about 1000 μl. Capacity for a capillary tube can be about 50 μl, about 55 μl, about 60 μl, about 65 μl, about 70 μl, about 75 μl, about 80 μl, about 85 μl, about 90 μl, about 95 μl, about 100 μl, about 110 μl, about 120 μl, about 130 μl, about 140 μl, about 150 μl, about 160 μl, about 170 μl, about 180 μl, about 190 μl, about 200 μl, about 225 μl, about 250 μl, about 275 μl, about 300 μl, 325 μl, about 350 μl, about 375 μl, about 400 μl, about 425 μl, about 450 μl, about 475 μl, about 500 μl, about 525 μl, about 550 μl, about 575 μl, about 600 μl, about 650 μl, about 700 μl, about 750 μl, about 800 μl, about 850 μl, about 900 μl, about 950 μl or about 1000 μl.

Furthermore, in various embodiments, the open end of a capillary tube can be designed for a friction fit, snap-on, screw top or other similar cap. Such caps can also be economically molded from any suitable plastic, for example, from a copolymer made from acrylonitrile, butadiene, and styrene (ABS), or polypropylene. Therefore, in various embodiments, once a sample is collected, the capillary assembly is removed, capped or not capped (capillary action preventing outflow) and coupled to another device or component via an adaptor (e.g., if screw top capillary can be screwed into other device, friction fit, etc.).

In one embodiment, a capillary tube (and cutting/piercing implement) used in a sampling device of the invention is disposable, or provided for a single use. In one example, a capillary tube with lancet implement is prepackaged (e.g., sterile packaging) and is capable of being pumped by a small vacuum pump with appropriate filters connecting it to the pump (which is permanent). In various embodiments of the invention, the capillary tube and lancet, (collectively referred to as “cartridge” or “assembly”) and/or sleeve are provided in one disposable assembly that couples to a housing unit which comprises additional components, such as a pump, microfluidic analysis component or microfluidic filter unit, or any other component necessary for analysis or processing of the sample (e.g., storage vessels in fluid communication with an inlet channel).

In some embodiments, the capillary assembly (capillary tube and cutting/piercing implement) are utilized to draw a sample, the assembly is removed from the impulse unit and coupled to another component or device, whereby such coupling is either through the distal (end fitting into impulse actuator adaptor/seat) end or the proximal end (end from which sample is collected). For example, the distal end can comprise a snap-on, screw type or luer lock which is capable of coupling to a complementary adaptor on a second component or device (e.g., microfluidic filter). In another example, the proximal end of the assembly is coupled to a second device/component through friction fit, snap-on or screw-type fitting. In one embodiment, a capillary and the piercing/cutting implement are in a fixed position. In another embodiment, the capillary and piercing/cutting implement can move independently (e.g., dual coaxial movement).

In one embodiment, the disposable cartridge couples to an inlet channel through an inlet port. In further embodiments, the components housed in the housing unit are also disposable. For example, a microfluidic card or filter unit can be swapped out of the housing unit. Furthermore, such disposable cartridges can contain filter units and absorbent pads which will reduce the likelihood of contamination of the ports which enable fluid communication between the capillary/lancet assembly and the downstream analysis and/or processing components present in the housing unit.

In addition, such filters and absorbent pads can also prevent contamination of ports within the housing unit which allow fluid communication between or amongst various components in the housing unit (e.g., storage vessels, microfluidic filters).

In various embodiments, capillary action alone is sufficient for a sample to flow into the capillary tube. However, in some embodiments, a vacuum tube/pump can be attached to the other end of a capillary tube directly or indirectly. For example, the capillary tube can be fitted with an adaptor which can couple the capillary tube the vacuum tube/pump tube with a collection vessel for collecting the sample. In some embodiments, various components can be placed in between the capillary tube and the vacuum/pump, such that the sample travels from the capillary tube to such one or more components. Such components include but are not limited to microfluidic filters, microfluidic analysis cartridges, sample collection vessels, sample tubes containing reagents necessary to perform biochemical and/or immunochemical assays.

In one embodiment, all components of the sampling device, including downstream analysis components are disposable. In further embodiments, such devices can be linked in series, and in fluid communication with each other and with the vacuum/pump, wherein the tubing connecting the last component to the pump contains one or more filters, as well as one or more absorbent material to prevent contamination of the filter. Such traps, filters and material are conventionally used in such vacuum/pumps for use with biologics.

In one embodiment, the optional vacuum shroud 103 (also referred to as “sleeve” herein) is used to ensure that a sample drawn from a subject does not splash or aerosolize, such as to become contaminated with the outside environment (e.g., air outside the shroud). For example, a sample such as blood is drawn but is prevented from contacting air or the outside environment. Furthermore, such a sleeve can prevent or reduce the likelihood of contamination or exposure to the surrounding environment (e.g., person(s) administering/operating the device).

In one embodiment, a sampling device of the invention is capable of use with a vacuum (e.g., pump) to provide back pressure and to draw a sample from the break in the skin. Furthermore, a vacuum can have one or more filters to prevent contamination of the pump. In some embodiments, capillary action alone is sufficient to draw a sample into a capillary tube of the invention.

In various embodiments, a sample obtained is any bodily fluid capable of being drawn into a capillary tube of the device of the invention. Such samples include but are not limited to blood, interstitial fluid, or both. Furthermore, samples can be obtained from any bodily cavity (e.g., buccal, nasal, vaginal, penal, anal), whereby a capillary tube of the invention can be administered to collect a sample via capillary flow. Therefore, in some embodiments, a disposable assembly is provided comprising a capillary tube, without a coaxial piercing/cutting implement.

Reagents for various samples can be selected as desired and coated onto a capillary tube's wall(s). Such reagents include anticoagulants, antibodies for removing or isolating target components present in blood and/or nucleic acid.

In various embodiments, the sampling device provides a first stage of a multiplexed system, which can include one or more additional components for analyzing or processing a sample.

Coupling to downstream components/methods (e.g., thermal cycler, microfluidic separation of blood components, centrifuge)

Pump

In various embodiments of the invention, a pump is operably linked to a sampling device of the invention. The term “operably linked” references the integration of the pump into the device so that when the pump is activated, negative or positive pressure can be applied to a sealed capillary tube. As described further herein, a capillary tube can form a seal or itself be encased in an outer sleeve that forms a seal, in which a vacuum can be obtained through activation of a pump operably linked to a device of the invention.

In various embodiments, the vacuum pump will also help in the extraction of blood as it will bring more blood to the tip of the finger by means of vacuum. The device will be pushed over the finger and the vacuum started. After a suitable time the capillary tube will be pushed up against the finger and a spring loaded device will push the metal pin, which is in the capillary tube towards the finger in order to make the incision. Blood will then flow up the capillary tube, either by means of capillary action alone, or through the action of a separate vacuum attached via a tube to the other end of the capillary tube after the spring loaded mechanism has been moved out of the way and a vacuum tube placed on the capillary tube, although in general this is not necessary as capillary action alone can be relied upon to move blood into the capillary tube.

The vacuum pump must be capable of providing a vacuum that will provide sufficient suction to stretch the portion of the skin in the region from which the sample of blood is to be extracted. Typically, the portion of stretched skin is raised a distance of 1 to 10 mm, preferably 3 to 5 mm, from the plane of the body part of which it is a portion. As the suction provided by the vacuum pump is stretching the appropriate portion of skin, the suction provided by the vacuum pump also causes the stretched portion to become engorged with blood. The level of suction provided must be sufficient to cause a relatively large volume of blood to become engorged at the point that the vacuum is applied. The vacuum pump must also be capable of providing sufficient suction to extract blood from the opening in the skin at a rate sufficient to extract at least 1 μL of blood within a period of five minutes. A vacuum pump that is suitable for the device of this invention can be a diaphragm pump, a piston pump, a rotary vane pump, or any other pump that will perform the required functions set forth previously.

Typically, the vacuum pump employs a self-contained permanent magnet DC motor. The vacuum pump provides a pressure of down to about −14.7 psig, and is more preferably operated at from about −3.0 psig to about −10.0 psig. The area of the skin subjected to vacuum preferably ranges up to about 50 cm², more preferably from about 0.1 to about 5.0 cm². The period of vacuum application prior to forming the opening in the skin, i.e., for increasing the availability of blood to the application site, preferably ranges up to about 5 minutes, preferably from about 1 to about 15 seconds. The period of vacuum application subsequent to forming the opening in the skin, i.e., for aiding in the extraction of blood from the unobstructed opening, preferably ranges up to about 5 minutes, preferably from about 1 to about 60 seconds. The vacuum provided by the vacuum pump can be continuous or pulsed. A continuous vacuum is preferred for the reason that it requires fewer components than does a pulsed vacuum. In further embodiments, where the coaxial assembly of the capillary tube-cutting/piercing implement is removed from the impulse housing unit (after a sample is obtained) and fitted to another component or device (e.g., microfluidic filter), such other component or device can also be fitted to a tube/portal so that a vacuum can be applied. For example, in one embodiment, the sample obtained flows into another component via the pump and/or through capillary flow alone.

It is also preferred that the level of vacuum applied and duration of application of vacuum not be so excessive that it causes the dermis to separate from the epidermis, which results in the formation of a blister filled with fluid. Vacuum pumps that are suitable for this invention are well-known to those of ordinary skill in the art and are commercially available; see U.S. Pat. No. 6,837,858 or T-Squared Manufacturing Company, USA.

In one embodiment, an impulse mechanism serves both to give a quick jab to the metal, plastic or glass incision rod 100 in the capillary tube 102, such as through a spring loaded mechanism, and/or gently pushing the capillary tube against the skin (e.g., finger 104) exactly around where the incision was made in order to collect the blood without loss. In one embodiment, a single impulse component pushes both the capillary and a cutting/piercing implement into the skin surface. In another embodiment, a single impulse component pushes a capillary into the skin surface, where the capillary lip (“sharp capillary”) is designed to break the skin surface to provide a sample, which flows into the capillary tube.

In one embodiment, two different spring impulses 101 can be used at the same time, one short, sharp jab to cut the skin, and a second softer impulse to push the glass against the skin. Such spring impulse components would function to extend the coaxial assembly of a capillary tube and a cutting/piercing implement. In further embodiments, such an assembly of the capillary-cutting/piercing implement can be manually or automatically removed from the impulse means housing unit 101 and coupled to another component or device for downstream analysis or processing.

In one embodiment, the capillary 102 tube is manually or automatically placed into another device and pressure (for instance through a connected pipe) can be used to transfer the blood out of the tube into another vessel, for instance into a microfluidic analysis system, or a microfluidic blood filter. For example, in one embodiment, a microfluidic filter will convert blood drawn from a subject from anti-coagulated blood to plasma.

In one aspect of the invention, a sampling device is provided comprising, a capillary tube and a lancing implement present inside said capillary tube, wherein said lancing element is capable of breaking a layer of skin sufficiently to draw a sample from a subject.

In one embodiment, a capillary tube comprises at least one end which is open or capable of being open. In one embodiment, the capillary tube comprises two open ends.

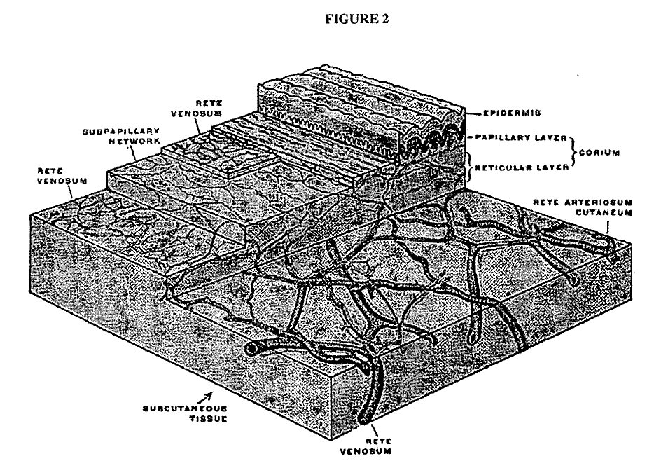

In one embodiment, a sampling device of the invention comprises at least one impulse component operably linked to said capillary tube and/or lancing implement. The impulse component can extend an implement for cutting or piercing the skin (e.g., pin, rod, wire, lancet) beyond an open end of a capillary tube, which is in contact with a skin surface FIG. 2. For example, where a capillary tube is placed in contact with a skin surface (e.g., heel, finger, toe), an open end of a capillary tube is placed in contact with the skin to produce a seal. This can be achieved manually (e.g., finger moved forward to contact capillary tube, such as after removing a protective cap on the capillary tube) or can be achieved automatically, where an impulse component (e.g., spring mechanism) is activated and extends the capillary forward until it firmly encounters resistance (e.g., where it comes into contact with a skin surface).

In one embodiment, a second impulse component moves the cutting or piercing implement forward to cut or pierce the skin. Such cutting or piercing is achieved by the cutting or piercing implement because the impulse element extends the implement beyond the lip of the capillary tube. Thus the implement comes into contact with a skin surface and cuts or piercing such a skin surface (e.g., FIG. 2). In various embodiments, the implement can be extend beyond the lip of a capillary tube by about 0.5 mm 1 mm, about 2 mm, about 3 mm, about 4 mm, about 5 mm. The length of the extension (i.e., puncture or cut depth) can be adjusted as desired by manually or automatically adjusting a mechanical stop which controls how deep the cutting/piercing implement can be extended.

Therefore, in various embodiments, the capillary tube and the cutting/piercing implement are coaxially situated and extended by at least two different coaxial actuators present in the housing unit 101. For example, one coaxial impulse means will extend a capillary tube to a length set by a mechanical stop, while a second coaxial impulse means extends a cutting/piercing implement to a set depth. In further various embodiments, such a capillary tube-cutting/piercing implement is a one-use assembly which is coupled to a impulse means housing 101 for sample collection (with our without a sleeve 103), and once a sample is collected the assembly is coupled to another device, chip or component (e.g., microfluidic filter, card, etc.). In one embodiment, the assembly is a coaxial assembly wherein the proximal end of the capillary tube is the end that is contacted with a skin surface, while the distal end of the capillary tube is the end that is coupled to the impulse housing. Furthermore, in various embodiments, the distal end is capped with a filter/absorbent material which prevent or eliminates the likelihood of a sample contaminating the impulse means.

Alternatively, in some embodiments, the distal end of the capillary tube provides a microfluidic channel in fluid communication with/through a port situated on the housing unit so as to provide fluid communication through the capillary tube, through a entry portal in the housing means, to a microfluidic channel in the housing means which is in fluid communication with one or more components (e.g., a microfluidic filter), devices, vessels or a combination.

In one embodiment, after a sample is obtained (e.g., cutting/piercing implement is retracted), the capillary tube can be inserted into a microfluidic filter or microfluidic reaction chip. The sample can be drawn into the additional component through capillary flow or can be drawn via a pump (manual, such as a air-bulb action, or automatic). Therefore, in various embodiments, the capillary-cutting/piercing implement is mobilizable from a sampling mode to a reaction/processing mode by virtue of being coupled to the impulse housing means 101 and being removed and coupled to another component. In one embodiment, the capillary tube-cutting/piercing assembly is a disposable coaxial unit.

In various embodiments, the impulse component is spring actuated. As one non-limiting example, generally a cylindrical portion is coupled to a cutting/piercing implement 100 connected to the distal end of the slide and a cylindrical extension or spring connector at the proximal end of the slide. A spring, such as a coil spring, is disposed in a bore present on the impulse housing unit 101. Spring is engaged between the holder and a lower end portion of the housing. The connector may be provided with a series of external integral bumps disposed in a spiral arrangement for threadedly receiving and holding the upper or distal end of coil spring. Therefore, a spring is threaded onto connector until the end of the spring engages an integral stop bump. An end plug can be provided with an integral extension or spring connector, where a cutting/piercing implement (e.g., along with the capillary) can be coupled. A spring is threaded onto connector until the end of the spring engages an integral stop bump. If desired, the spring connectors may be provided with suitable screw threads instead of the series of bumps or the ends of the spring may be fixed to those connectors by other suitable means. In various embodiments, the sampling device of the invention can incorporate an impulse component such as any conventional spring actuated impulse means or other means for extending the capillary and/or cutting (or piercing) implement. Some examples of such impulse components are disclosed in U.S. Pat. Nos. 4,503,856; 4,653,513; 4,517,978; 5,879,311; 6,071,294; 6,155,952; and 6,612,111.

In various embodiments, a capillary tube is coated with one or more reagents. Such reagents include anticoagulants, antibodies, nucleic acids, aptamers and/or agents designed to directly or indirectly bind one or more components present in the sample being drawn. For example, anticoagulants can prevent coagulation of a blood sample. Furthermore, agents designed to bind particular components present in a blood sample can function to reduce, deplete or eliminate components which can interfere with downstream processes or analysis of the sample.

In various embodiments of the invention, different size capillary tubes with a cutting or piercing implement can be coupled to the housing unit for one or more impulse components. As such, based on the size of the capillary tube, various different volumes of a sample can be draw. In various embodiments, a capillary tube has a capacity of from about 20 ul to about 1000 ul, as described herein.

In another aspect of the invention, a sampling device comprises a sleeve 103 into which said capillary tube is inserted and comprising an open end which forms a seal around a subject's skin

In one embodiment, a sampling device of the invention comprising at least two impulse components, wherein one of said impulse components extends the capillary tube and a second impulse component extends the cutting/lancing implement. In various embodiments, an impulse component is capable of impelling said lancing implement to a position of about 0.5 mm, 1 mm, 2 mm, 3 mm, 4 mm, 5 mm, or 6 mm beyond end of said capillary tube which is in contact with a skin surface. In some embodiments, the skin surface is on a finger, a heel or a toe. A cut or a piercing through skin is achieved sufficiently so to penetrate the capillary rich layer (FIG. 2). Therefore, depending on the surface from which a sample is to be drawn, a mechanical stop can be set for the impulse means driving the cutting/piercing implement. The mechanical stop can be set manually or automatically to provide a piercing/cutting depth as described herein. For example, if sampling through a well calloused heel, the depth may be set to 3 to 4 mm, while sampling from a finger tip may be about 0.5 mm.

In one aspect of the invention, a method is provided for obtaining a sample from a subject comprising: contacting skin of said subject with a sampling device comprising a capillary tube comprising a lancing implement to cut or pierce said subject's skin, breaking the subject's skin with said lancing implement and flowing blood from said break into said capillary.

In one embodiment, a sampling device further comprises at least one impulse component, said impulse component operating to impel said capillary and/or lancing implement to said subject's skin, wherein said contacting comprises actuating (or activating) said impulse device. In one embodiment, at least one impulse component is activated. In another embodiment, at least two impulse components, wherein one of said one or more impulse components actuates said capillary and wherein a second of said one or more impulse components actuates said lancing implement.

In some embodiments, such capillary is coated with one or more reagents, such as anti-coagulants, an antibody, an aptamer, nucleic acid molecule or an agent designed to bind (directly or indirectly) one or more components present in the sample.

In one embodiment, a method for obtaining a sample comprises utilizing a sampling device of the invention comprising a sleeve which surrounds said subject's skin. In further embodiments, the skin surrounded by a sleeve is on an appendage, such as a finger, toe or heel.

In some embodiments, a method for obtaining a sample comprises utilizing a sampling device is capable of holding from about 20 ul to about 1000 ul of the sample.

In one embodiment, a method for obtaining a sample comprises utilizing a sampling device which provides vacuum suction as described herein. Of course it should be understood that in some circumstances, a sample can be drawn through capillary action alone.

In other embodiment, a capillary tube itself is designed so that the capillary lip in contact with a skin surface will cut the skin to draw a sample there from. Therefore, such a capillary tube can be used to draw a sample and subsequently be coupled to any other component or device. For example, in one embodiment, such a capillary tube is used to draw a sample and is coupled to a microfluidic chip, microfluidic filter, or reaction tube comprising reagents necessary to perform downstream analysis (e.g., PCR, immunobiochemistry, etc.).

In various embodiments, a capillary tube of the invention can be used to collect a sample without cutting or piercing a surface. For example, such a capillary tube can be used to collect a buccal, nasal, or vaginal sample where there are ample bodily fluids available (e.g., saliva). If moisture is needed artificially, any conventional means to provide additional moisture can be utilized to obtain a sample (e.g., saline nasal drops). In further embodiments, such a capillary can be used to obtain a sample from an abscess, wound or other skin trauma to obtain a sample. For example, such a capillary tube can be used to obtain a sample from a skin lesion and the capillary can be subsequently coupled to a microfluidic card to process the sample (e.g., reagents necessary to provide access to nucleic acids and/or primers and enzymes necessary to conduct PCR amplification), and where the microfluidic card is subsequently coupled to a PCR machine. In one embodiment, such a capillary tube is glass.

What is claimed is:

1. A sampling device comprising: a capillary tube and a lancing or cutting implement present inside said capillary tube, wherein said lancing element is capable of breaking a layer of skin sufficiently to draw a sample from a subject.

2. The sampling device of claim 1, wherein said capillary tube comprises at least one end which is open or capable of being open.

3. The sampling device of claim 1, further comprising at least one impulse component operably linked to said capillary tube and said lancing or cutting implement.

4. The sampling device of claim 1, wherein said capillary tube is coated with one or more reagents.

5. The sampling device of claim 1, wherein said capillary tube has a capacity of from about 20 μl to about 1000 μl.

6. The sampling device of claim 1, further comprising a sleeve into which said capillary tube is inserted and comprising an open end which forms a seal around a subject's skin.

7. The sampling device of claim 1, wherein said breaking is through a cut or a piercing of said skin.

8. The sampling device of claim 3, wherein said impulse component is spring-actuated, electromagnetic or piezoelectric.

9. The sampling device of claim 4, wherein said one or more reagents is an anticoagulant(s).

10. The sampling device of claim 6, comprising at least two impulse components, wherein one of said impulse components extends said capillary tube and another of said impulse components extends said lancing or cutting implement.

11. The sampling device of claim 6, wherein said device is capable of providing vacuum suction in said capillary.

12. The sampling device of claim 10, wherein said second impulse component is capable of impelling said lancing implement to a position of about 0.5 mm, 1 mm, 2 mm, 3 mm, 4 mm, 5 mm, or 6 mm outside an open end of said capillary tube.

13. The sampling device of claim 11, wherein said subject's skin is on a finger, a heel or a toe.

14. A method of obtaining a sample from a subject comprising: contacting skin of said subject with a sampling device comprising a capillary tube comprising a cutting or lancing implement to said subject's skin, breaking the subject's skin with said lancing implement and flowing blood from said break into said capillary.

15. The method of claim 14, wherein sampling device further comprises at least one impulse component, said impulse component operating to impel said capillary and cutting or lancing implement to said subject's skin, wherein said contacting comprises actuating said impulse device.

16. The method of claim 15, wherein said at least one impulse component extends the cutting or piercing implement in a jabbing motion to cut or pierce the skin.

17. The method of claim 15, comprising at least two impulse components, wherein one of said one or more impulse components actuates said capillary and wherein a second of said one or more impulse components actuates said lancing implement.

18. The method of claim 14, wherein said capillary is coated with one or more reagents.

19. The method of claim 14, wherein said capillary is coated with one or more anti-coagulants.

20. The method of claim 14, wherein said sampling device further comprises a sleeve which surrounds said subject's skin.

21. The method of claim 14, wherein said sampling device is capable of holding from about 20 ul to about 1 ml of said sample.

22. The method of claim 20, wherein said skin surrounded by said sleeve comprises a subject's appendage.

23. The method of claim 20, wherein said device provides vacuum suction.

24. A method of obtaining a sample from a subject comprising: contacting skin of said subject with a sampling device comprising a capillary tube comprising an edge for breaking the subject's skin to allow blood flow into said capillary.

25. The method of claim 24, wherein said capillary tube is pushed forward into the skin by an impulse component.
